(19) United States
(12) Patent Application Publication
(10) Pub. No.: US 2007/0129690 A1
(43) Pub. Date: Jun. 7, 2007

(54) Catheter with polymeric coating

(76) Inventors: Joel Rosenblatt, Pottstown, PA (US); Jeff Vitullo, Pottstown, PA (US); Mike Bouchard, Bernville, PA (US)

Correspondence Address: AMSTER, ROTHSTEIN & EBENSTEIN LLP, 90 PARK AVENUE, NEW YORK, NY, 10016, US

(21) Appl. No.: 11/293,056

(22) Filed: Dec. 2, 2005

(51) Int. Cl. A61M 25/00; A61M 5/32

(52) U.S. Cl. 604/265; 424/422

(57) ABSTRACT

A catheter including a main body having a first end, a second end, a lumen extending between the first end and the second end, a first section located proximal the first end of the main body and second section located proximal the second end of the main body. An inhibitory polymer is disposed at the first section. The inhibitory polymer includes one or more members selected from the group consisting of antiproliferatives, antithrombotics, thrombolytics, and fibrinolytics. An antimicrobial agent is disposed at the second section. The main body has a length such that when the catheter is at least partially implanted the first end accesses a body vessel and at least a portion of the second section is disposed within a subcutaneous space of a patient.

### TECHNICAL FIELD

The present invention relates to medical devices suitable for at least partial implantation into a body. More specifically, the present invention relates to catheters having therapeutic agents.

### BACKGROUND

When implanted, medical devices, such as catheters, are placed in intimate contact with a variety of cells, tissues, and body systems, thereby presenting an opportunity for infection. In essence, catheters provide a path from the external environment into the body along which microorganisms can colonize, and eventually produce an infection. The establishment of an infection can require intervention, such as treatment with a therapeutic agent or even mechanical manipulation of the medical device to remove the microorganisms. Even worse, the infection may require removal and replacement of the medical device. Ultimately, the presence of an infection may outweigh the benefits of the implantation.

Catheters may also cause additional problems related to coagulation of blood. In particular, it is well known that when blood comes into contact with a surface other than the natural wall of a blood vessel, the activation of certain circulating substances results in the coagulation of the blood. If thrombi are formed on portions of the surface which contact blood flow, there is a risk that the thrombi will be released and cause serious blood circulation disturbances called thrombosis.

Thus, there is a need for a catheter that provides both effective protection against infection as well as anti-coagulant properties.

### SUMMARY OF THE INVENTION

A catheter according to an exemplary embodiment of the invention includes a main body having a proximal portion, a distal portion and a lumen extending between the proximal portion and the distal portion of the main body. An antimicrobial agent is disposed at the proximal portion of the main body. An inhibitory polymer is disposed at the distal portion of the main body. The inhibitory polymer includes one or more members selected from the group consisting of antiproliferatives, antithrombotics, thrombolytics, and fibrinolytics.

A catheter according to another exemplary embodiment of the invention includes a main body having a first end, a second end, at least one lumen extending between the first end and the second end, a first section proximal the first end of the main body, and a second section proximal the second end of the main body. An inhibitory polymer is disposed at the first section. The inhibitory polymer includes one or more members selected from the group consisting of antiproliferatives, antithrombotics, thrombolytics, and fibrinolytics. An antimicrobial agent is disposed at the second section. The main body has a length such that when the catheter is at least partially implanted the first end accesses a body vessel and at least a portion of the second section is disposed within a subcutaneous space of a patient.

These and other features of this invention are described in, or are apparent from, the following detailed description of various exemplary embodiments of this invention.

### BRIEF DESCRIPTION OF THE DRAWINGS

Various exemplary embodiments of this invention will be described in detail, with reference to the following figures, wherein:

FIG. 1 shows a catheter according to an exemplary embodiment of the invention;

FIG. 2 shows the catheter of FIG. 1 in use;

FIG. 3 shows a catheter according to another exemplary embodiment of the invention;

FIG. 4 shows a portion of a catheter according to another exemplary embodiment of the invention; and

FIG. 5 shows a catheter according to another exemplary embodiment of the invention.

### DETAILED DESCRIPTION OF PREFERRED EMBODIMENTS

The various exemplary embodiments of the present invention are drawn to a catheter including an antimicrobial agent disposed at a first section of the catheter and an inhibitory polymer disposed at a second section of the catheter. As used herein, the term “disposed” means that a substance is positioned at least at the surface of the catheter by any suitable means, such as, for example, by coating the surface with the substance or by mixing the substance with the catheter material.

The term “inhibitory polymer” as used herein is meant to encompass any polymer that exhibits therapeutic properties, such as, for example, anticoagulant or antithrombotic properties. The present invention is not meant to be limited to any specific type of catheter, and the catheter structures described herein are intended to be merely exemplary. It should be appreciated that the therapeutic agents and polymeric coatings described herein can be applied to any type of known catheter design.

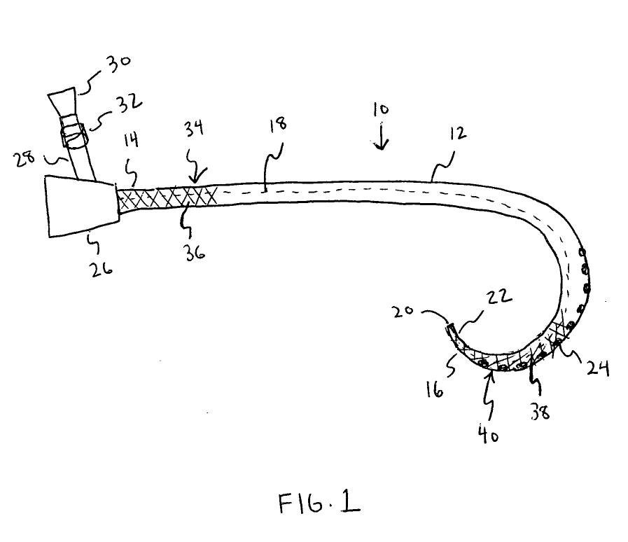

FIG. 1 shows a catheter 10 according to an exemplary embodiment of the present invention. The catheter 10 includes a main body 12 having circular cross-section of substantially uniform diameter. The main body 12 includes a proximal end 14 and a distal end 16. A lumen 18 extends through the main body 12 and exits through a port 20 at the distal end 16 of the main body 12. The main body 12 includes a tapered distal tip 22 and a plurality of longitudinally spaced ports or openings 24 are formed in the main body 12 at axially spaced locations proximal to the distal end 16. Each opening 24 directly communicates with the lumen 18. A hub 26 may be affixed to the proximal end of the main body 12 for connection to suitable drainage equipment, such as a drainage bag or a suction device. The catheter 10 may also include a branch line 28 for the purpose of, for example, infusion or sampling without disconnection of the drainage equipment. The branch line 28 may be fitted with a luer fitting 30 and a clamp 32 which is used to close off branch line 28 when not in use.

The catheter main body 12 may be made of any suitable biocompatible material, such as, for example, polyurethane. Also, in embodiments, the main body 12 may be heat set in a curved configuration for proper insertion into a body cavity.

In the present embodiment, an antimicrobial agent 36 is coated over a proximal region 34 of the main body 12 adjacent to the proximal end 14. As used herein, the term “antimicrobial agent” means any agent that has killing or growth inhibiting effects on one or more microorganisms. In exemplary embodiments of the invention, the antimicrobial agent 36 may be impregnated or agent dispersed into the proximal region 34. Suitable classes of antimicrobials include antibiotics, disinfectants, and antiseptics. In a preferred embodiment, the antimicrobial agent 36 includes one or more antibiotics having activity against the common microorganisms associated with colonization and/or infection with indwelling cannulae. Different antimicrobial agents can be used with the present invention. Examples include, but are not limited to, a guanidium (e.g., chlorhexidine, alexidine, and hexamidine), a biguanide, a bipyridine (e.g., octenidine), a phenoxide antiseptic (e.g., colofoctol, chloroxylenol, and triclosan), an alkyl oxide, an aryl oxide, a thiol, an aliphatic amine, an aromatic amine and halides such as F⁻, Br⁻and I⁻, and salts thereof. Additional examples include bismuth, gendine, genlenol, genlosan, genfoctol, silver sulfadiazine, chlorhexidine-silver sulfadiazine, chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an antimicrobial dye, and antimicrobial mixtures containing carbon and platinum. The antimicrobial dye can be, for example, a triarylmethane dye, a monoazo dye, a diazo dye, an indigoid dye, a xanthene dye, a fluorescein dye, an anthraquinone dye or a quinoline dye. More specific examples of dyes include gentian violet, crystal violet, ethyl violet, brilliant green, and methylene blue. Furthermore, different antibiotics or mixtures of antibiotics can be used with the present invention. A preferred mixture of antibiotics inhibits bacterial growth by different mechanisms, e.g., a DNA or RNA replication inhibitor combined with a protein synthesis inhibitor. Examples of agents that inhibit bacteria by inhibiting DNA or RNA replication include rifampicin, taurolidone, 5-fluorouracil, and Adriamycin. Examples of agents that inhibit protein synthesis include tetracyclines, e.g. minocycline, and clindamycin. Another category of an antimicrobial agent is quorum sensing inhibitors such as inhibitors of derivatives of Autoinducer 1 (N-acyl homoserine lactone) and Autoinducer 2 (furanosyl borate diester), inhibitors of their receptors, and inhibitors of the genes and kinases involved in their upregulation. Examples of quorum sensing inhibitors include furanones, including halogenated furanones. Still another category of an antimicrobial agent is a host-defense protein or peptide, such as an aminosterol or a magainin, or a mimetic thereof. Additional examples of antimicrobial agents can be found, e.g., in U.S. Pat. Nos. 5,221,732, 5,643,876, 5,840,740, 6,303,568, 6,388,108, and 6,875,744, in U.S. Patent Application Publication No. 2003/0078242, and in PCT International Publication No. WO 2004/099175, the contents of which are incorporated by reference. Preferably, the antimicrobial agent contains chlorhexidine (including the free base and salts thereof and mixtures of the free base and salts).

The antimicrobial agent 36 may include a combination of two or more antimicrobials. In these embodiments, the two or more antimicrobials can be located in or on discrete locations within the proximal region 34, or the two or more antimicrobials can be blended together and uniformly distributed within or on the proximal region 34

An inhibitory polymer 38 is coated over a distal region 40 of the main body 12 adjacent to the distal end 16. The inhibitory polymer is preferably any suitable polymer that provides anticoagulant, anti-thrombotic, thrombolytic, fibrinolytic, or antiproliferative properties, and preferably resists protein deposition. The inhibitory polymer 38 is preferably hydrophilic. Specific examples of suitable inhibitory polymers include polyethylene glycol (PEG), polyethylene oxide (PEO), polyvinyl pyrrolidone (PVP), polyvinyl alcohol (PVA) and phosphoryl choline (PC). Alternatively, the inhibitory polymer may also be hydrophobic, such as, for example, fluorinated polymers including polytetrafluoroethylene (PTFE), hexafluoropropene (HFP), polyvinylidine difluoride (PVDF), or fluorinated ethylene-propylene (FEP). Also, the inhibitory polymer may include degradable polymers that release bioactive agents, such as, for example, nitric oxide releasing polymers or polyaspirin. See, for example, Parzuchowski, Pawel G., Frost, Megan C., and Meyerhoff, Mark E., “Synthesis and Characterization of Polymethacrylate-Based Nitric Oxide Donors”, J. Am. Chem. Soc. 2002, 124, 12182-12191. As another example, polymers made with pendant diazenium diolate functional groups that donate nitric oxide when exposed to moisture may be used. See, for example, Saavedra, Joseph E.and Keefer, Larry K., “Nitrogen-Based Diazeniumdiolates: Versatile Nitric Oxide-Releasing Compounds for Biomedical Research and Potential Clinical Applications”, J. Chem. Educ. 2002, 79(12), 1427-1434.

Coatings incorporating PEO and isocyanates are known in the art (U.S. Pat. Nos. 5,459,317, 4,487,808 and 4,585,666 to Lambert; and U.S. Pat. No. 5,558,900 to Fan et al.). In addition, polyols may be incorporated into such PEO/isocyanate coatings to produce a crosslinked polyurethane (PU) network entrapping the PEO (U.S. Pat. Nos. 5,077,352 and 5,179,174 to Elton). PEO may also be combined with structural plastic having a high molecular weight to produce a coating with reduced friction (U.S. Pat. No. 5,041,100 to Rowland). In a preferred embodiment of the invention, the inhibitory polymer includes polyethylene oxide and the antimicrobial agent includes chlorhexidine.

PVP may be used as a coating alone or in combination with other polymers. One such coating is a PVP-polyurethane interpolymer (U.S. Pat. Nos. 4,100,309 and 4,119,094 to Micklus et al.). Another such coating is composed of hydrophilic blends of PVP and linear preformed polyurethanes (U.S. Pat. No. 4,642,267 to Cresy). In addition, PVP may be incorporated into a PU network by combining a polyisocyanate and a polyol with a PVP solution (U.S. Pat. Nos. 5,160,790 and 5,290585 to Elton). Still another such coating may be composed of two layers: a primer and a top coat. The primer coat may be a polyurethane prepolymer containing free isocyanate groups, while the top coat may be a hydrophilic copolymer of PVP and a polymer having active hydrogen groups, such as acrylamide (U.S. Pat. No. 4,373,009 to Winn).

Hydrophilic polyurethanes may also be used as the inhibitory polymer 38. For example, the coating may be composed of polyurethane hydrogels containing a random mixture of polyisocyanates and a polyether dispersed in an aqueous liquid phase (U.S. Pat. No. 4,118,354 to Harada et al.). Polyurethanes may also be used as coatings in compositions containing chain-extended hydrophilic thermoplastic polyurethane polymers with a variety of hydrophilic high molecular weight non-urethane polymers (U.S. Pat. No. 4,990,357 to Karkelle et al.).

PC in particular has proven to be effective in providing an anti-thrombogenic coating. Such coatings are disclosed in U.S. Pat. No. 5,658,561 to Nakabayashi et al., U.S. Pat. No. 6,673,883 to Rowan, U.S. Pat. No. 5,705,583 to Bowers et al., U.S. Pat. No. 6,090,901 to Bowers et al. and EP 0593561, the disclosures of which are incorporated by reference herein in their entirety.

It should be appreciated that the inhibitory polymer may be disposed at sections of the catheter by any suitable means, preferably by coating over the catheter surface or by blending with the material used to form the catheter. For example, block polymers that migrate to the surface after being blended with the catheter material, such as, for example, polyurethane-PEO or polyurethane fluorinated block copolymers, or that degrade and release active agents to the surface, may be used.

Other examples of suitable inhibitory polymers may include polymers that sequester or bind antithrombogenic factors from circulating blood, as disclosed in, for example, U.S. Patent Application Publication 2003/0185870A1, the contents of which are incorporated herein by reference. Also, polymers that have the ability to catalyze a therapeutic effect from latent effectors circulating in the blood may be used, such as, for example, Cu(II) containing ligands that generate nitric oxide from endogenous nitrite and nitrosothiols, as disclosed in U.S. Patent Application Publication 2002/0115559A1, the contents of which are incorporated herein by reference. See also, B. Oh and M.E. Meyerhoff, “Spontaneous Generation of Nitric Oxide from Nitrosothiols at Interface of Polymeric Films Doped with Lipophilic Copper(II) Complex”, J. Am. Chem. Soc. 2003, 125, 9552-3.

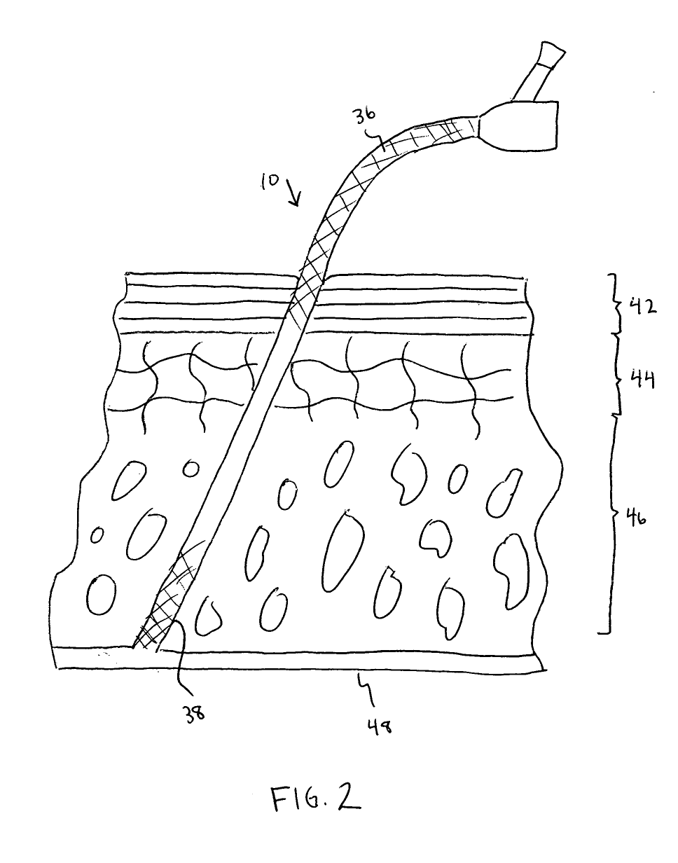

As shown in FIG. 2, when in use, the catheter 10 traverses the skin of a patient through the epidermis 42, the derma 44 and the subcutaneous layer 46 to a vessel 48. Thus, the therapeutic agent 36, coated over the proximal region 34 of the catheter 10, is able to provide protection against infection at the point where the catheter 10 enters the epidermis 42 and through the subcutaneous layer 46, while the inhibitory polymer 38, coated over the distal region 40 of the catheter 10, is able to provide suitable inhibitory effects below the subcutaneous layer 46 and within the vessel 48.

In an alternative embodiment, the antimicrobial agent 36 may be coated over the entire main body 12 of the catheter 10, rather than just over the proximal region 34. Thus, the entire catheter 10 may be provided with protection against infection. In other embodiments, the entire main body 12 may be coated with the inhibitory polymer 38. The antimicrobial agent 36 may be coated over the inhibitory polymer 38, or vice versa. In still other embodiments of the invention, the antimicrobial agent 36 may be coated over the hub 26 of the catheter 10 as well as the proximal region 34 of the main body 12.

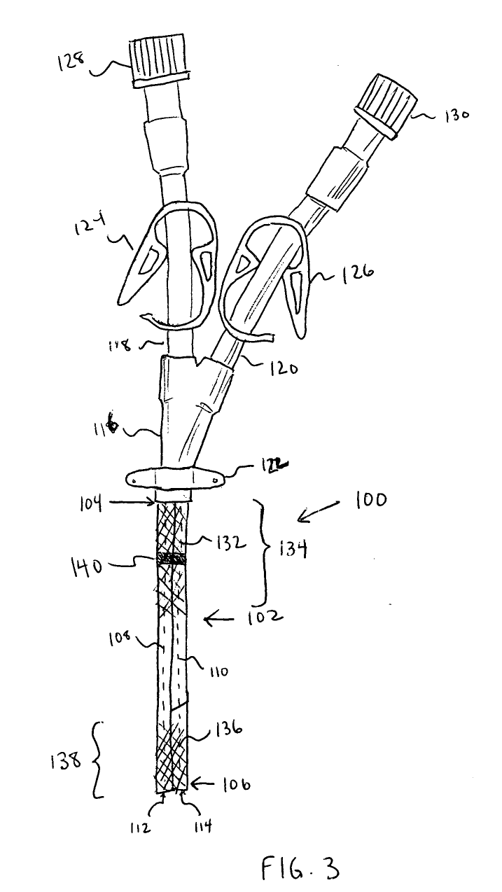

FIG. 3 shows a catheter 100 according to another exemplary embodiment of the present invention. The catheter 100 is a dialysis catheter, including a main body 102 having a proximal end 104 and a distal end 106. First and second lumens 108, 110 extend through the main body 102 and exit through respective ports 112, 114. The proximal end 104 of the catheter main body 102 is secured to a connector hub 116. A first connector tube 118 and a second connector tube 120 extend from the connector hub 116. The connector hub 116 couples the first connector tube 118 to the first lumen 108 for communication therewith, and couples the second connector tube 120 to the second lumen 110 for communication therewith. A suture wing 122 may be rotatably secured to the connector hub 116 to allow the connector hub 116 to be secured to the patients skin. In addition, a pair of clamps 124 and 126 may be secured over the connector tubes 118 and 120, respectively, for selectively closing off the connector tubes 118, 120 before and after each hemodialysis procedure. A pair of luer lock connector fittings 128 and 130 are secured to the free ends of the connector tubes 118 and 120, respectively, to allow the catheter 100 to be interconnected with fluid infusion lines, aspiration lines, or with the blood inlet and blood return ports of a hemodialysis machine. In the latter case, the first lumen 108 is coupled, via first connector tube 118 and luer lock fitting 128, to an aspiration port of a hemodialysis machine to withdraw blood containing toxins from a blood vessel; and the second lumen 110 is coupled, via second connector tube 120 and luer lock fitting 130, to a cleaned blood return port of the hemodialysis machine to return cleaned blood to the blood vessel. The catheter 100 may also include a stabilizing cuff 140 affixed to an outer portion of the catheter near the proximal end 104.

As in the previous embodiment, an antimicrobial agent 132 is coated over a proximal region 134 of the main body 102 adjacent to the proximal end 104, and an inhibitory polymer 136 is coated over a distal region 138 adjacent to the distal end 106. In exemplary embodiments of the invention, the antimicrobial agent 132 may be impregnated or agent dispersed into the proximal region 134. The antimicrobial agent 132 may be one or more of the antimicrobial agents previously listed herein. The inhibitory polymer is preferably any suitable polymer that provides anticoagulant, anti-thrombotic, thrombolytic, fibrinolytic, or antiproliferative properties, such as those polymers previously listed herein. Also, in another embodiment, the antimicrobial agent 132 may be coated over the entire main body 102 of the catheter 100, rather than just over the proximal region 134. Alternatively, the entire main body 102 may be coated with the inhibitory polymer 136. The antimicrobial agent 132 may be coated over the inhibitory polymer 136, or vice versa. In still other embodiments, the antimicrobial agent 132 may be coated over the connector hub 116 and/or the connector tubes 118, 120 as well as the proximal region 134 of the main body 102.

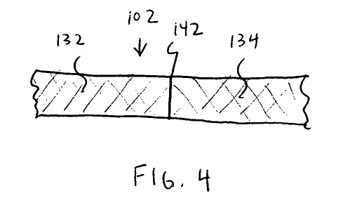

In various exemplary embodiments of the invention, the area of the catheter coated with the antimicrobial agent may be visually differentiated from the area coated with inhibitory polymer. For example, as shown in FIG. 4, a separator 142 may be used to differentiate the area with the antimicrobial agent 132 from the area with the inhibitory polymer 134. In the embodiment shown in FIG. 4, the separator 142 is a marking that may be printed on the main body 102. Alternatively, each area may have a different color, or the areas may be separated by a reduced diameter portion of the main body 102. Indicating the different areas of that catheter may aid fabrication and implantation procedures.

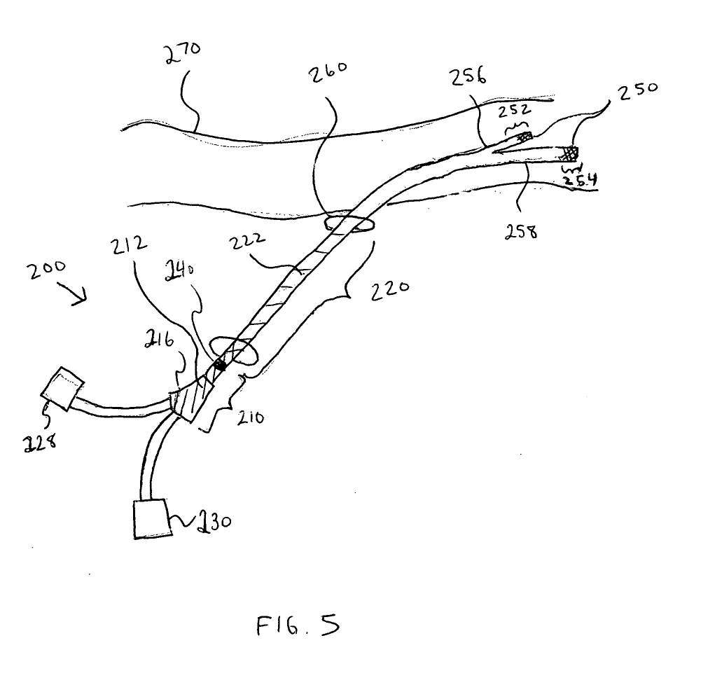

FIG. 5 shows a catheter 200 according to another exemplary embodiment of the invention inserted into a vessel 270 through a venotomy site 260. The catheter 200 has generally the same structure as the catheter 100, including luer lock fittings 228, 230, connector hub 216 and a cuff 240. An antimicrobial agent 212 is disposed at a proximal region of the catheter 200, including at least the region from the hub 216 to the cuff 240. A first inhibitory polymer 222, preferably an antiproliferative, is disposed at an intermediate region 220 extending from at least the cuff to the venotomy site 260. A third inhibitory polymer 250, preferably an antithrombotic, thrombolytics or fibrinolytic, is disposed at the respective distal end regions 252 and 254 of first and second lumens 256 and 258. Thus, in the present embodiment, the catheter includes essentially three zones; an antimicrobial zone, an antiproliferative zone and a antithrombotic zone. In other embodiments, a non-polymeric antiproliferative may be disposed at the intermediate region 220 of the catheter 200, such as, for example, chemotherapeutics such as palitaxel and DNA alkylating agents as well as mTOR inhibitors such as rapamycin and rapamycin analogues.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A catheter comprising:
a main body having a proximal portion, a distal portion and a lumen extending between the proximal portion and the distal portion of the main body;
an antimicrobial agent disposed at the proximal portion of the main body; and
an inhibitory polymer disposed at the distal portion of the main body, the inhibitory polymer comprising one or more members selected from the group consisting of antiproliferatives, antithrombotics, thrombolytics, and fibrinolytics.

2. The catheter of claim 1, wherein the entire main body is disposed with the inhibitory polymer.

3. The catheter of claim 2, wherein the inhibitory polymer is disposed over the antimicrobial agent.

4. The catheter of claim 2, wherein the antimicrobial agent is disposed over the inhibitory polymer.

5. The catheter of claim 1, wherein the entire main body is disposed with the antimicrobial agent.

6. The catheter of claim 5, wherein the inhibitory polymer is disposed over the antimicrobial agent.

7. The catheter of claim 5, wherein the antimicrobial agent is disposed over the inhibitory polymer.

8. The catheter of claim 1, wherein the inhibitory polymer comprises polyethylene glycol, polyethylene oxide, polyvinyl pyrrolidone, polyvinyl alcohol, polytetrafluoroethylene, hexafluoropropene, polyvinylidine difluoride, fluorinated ethylene-propylen, a nitric oxide releasing polymers, a polymer that catalyzes conversion of endogenous inhibitory substrates or a polymer that sequesters endogenous antithrombogenic agents.

9. The catheter of claim 1, wherein the inhibitory polymer comprises polyethylene oxide.

10. The catheter of claim 1, wherein the antimicrobial agent comprises any one or more of a guanidium, a biguanide, a bipyridine, a phenoxide antiseptic, an alkyl oxide, an aryl oxide, a thiol, an aliphatic amine, an aromatic amine, colofoctol, chloroxylenol, triclosan, gendine, genlenol, genlosan, genfoctol, octenidine, chlorhexidine, alexidine, hexamidine, silver sulfadiazine, chlorhexidine-silver sulfadiazine, chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an aminosterol, a magainin, a furanone, a halogenated furanone, a triarylmethane dye, a monoazo dye, a diazo dye, an indigoid dye, a xanthene dye, a fluorescein dye, an anthraquinone dye, a quinoline dye, gentian violet, crystal violet, ethyl violet, brilliant green, methylene blue, rifampicin, taurolidone, 5-fluorouracil, Adriamycin, a tetracycline, minocycline, clindamycin, rifampin-minocycline, and salts thereof.

11. The catheter of claim 1, wherein the antimicrobial agent comprises chlorhexidine.

12. The catheter of claim 11, wherein the inhibitory polymer comprises polyethylene oxide.

13. The catheter of claim 1, wherein the proximal portion comprises a hub.

14. The catheter of claim 1, wherein the proximal portion comprises at least one connector tube.

15. The catheter of claim 1, wherein the proximal portion comprises a cuff.

16. The catheter of claim 1, wherein the proximal portion comprises a hub and a cuff, and the antimicrobial agent extends at least from the hub to the cuff.

17. The catheter of claim 1, wherein the proximal portion comprises a cuff, and the inhibitory polymer comprises an antiproliferative and extends at least from the cuff to the venotomy.

18. The catheter of claim 1, wherein the distal portion terminates at a distal tip, and the inhibitory polymer comprises an antithrombotic, a thrombolytics or fibrinolytic and is disposed at the distal tip.

19. The catheter of claim 1, wherein the proximal portion comprises a hub and a cuff, the antimicrobial agent extends at least from the hub to the cuff, and the inhibitory polymer comprises an antiproliferative that extends at least from the cuff to the venotomy and an antithrombotic, a thrombolytics or fibrinolytic that is disposed at a distal tip of the distal portion.

20. A catheter comprising:
a main body having a first end, a second end, at least one lumen extending between the first end and the second end, a first section proximal the first end of the main body, and a second section proximal the second end of the main body, an inhibitory polymer disposed at the first section, the inhibitory polymer comprising one or more members selected from the group consisting of antiproliferatives, antithrombotics, thrombolytics, and fibrinolytics, and an antimicrobial agent disposed at the second section,
the main body having a length such that when the catheter is at least partially implanted the first end accesses a body vessel and at least a portion of the second section is disposed within a subcutaneous space of a patient.

21. The catheter of claim 20, wherein the entire main body is disposed with the inhibitory polymer.

22. The catheter of claim 21, wherein the inhibitory polymer is disposed over the antimicrobial agent.

23. The catheter of claim 21, wherein the antimicrobial agent is disposed over the inhibitory polymer.

24. The catheter of claim 20, wherein the entire main body is disposed with the antimicrobial agent.

25. The catheter of claim 24, wherein the inhibitory polymer is disposed over the antimicrobial agent.

26. The catheter of claim 24, wherein the antimicrobial agent is disposed over the inhibitory polymer.

27. The catheter of claim 20, wherein the inhibitory polymer comprises polyethylene glycol, polyethylene oxide, polyvinyl pyrrolidone, polyvinyl alcohol, polytetrafluoroethylene, hexafluoropropene, polyvinylidine difluoride, fluorinated ethylene-propylen, a nitric oxide releasing polymer, a polymer that catalyzes conversion of endogenous inhibitory substrates or a polymer that sequesters endogenous antithrombogenic agents.

28. The catheter of claim 20, wherein the antimicrobial agent comprises any one or more of a guanidium, a biguanide, a bipyridine, a phenoxide antiseptic, an alkyl oxide, an aryl oxide, a thiol, an aliphatic amine, an aromatic amine, colofoctol, chloroxylenol, triclosan, gendine, genlenol, genlosan, genfoctol, octenidine, chlorhexidine, alexidine, hexamidine, silver sulfadiazine, chlorhexidine-silver sulfadiazine, chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an aminosterol, a magainin, a furanone, a halogenated furanone, a triarylmethane dye, a monoazo dye, a diazo dye, an indigoid dye, a xanthene dye, a fluorescein dye, an anthraquinone dye, a quinoline dye, gentian violet, crystal violet, ethyl violet, brilliant green, methylene blue, rifampicin, taurolidone, 5-fluorouracil, Adriamycin, a tetracycline, minocycline, clindamycin, rifampin-minocycline, and salts thereof.

29. The catheter of claim 20, wherein the antimicrobial agent comprises chlorhexidine.

30. The catheter of claim 29, wherein the inhibitory polymer comprises polyethylene oxide.

31. The catheter of claim 20, wherein the catheter is a dialysis catheter.

32. The catheter of claim 31, wherein the at least one lumen comprises an intake lumen and an outlet lumen.

33. The catheter of claim 20, wherein the main body further comprises a separator that separates the first section from the second section.

34. The catheter of claim 33, wherein the separator is a marking.

35. The catheter of claim 20, wherein the second section comprises a hub.

36. The catheter of claim 20, wherein the second section comprises a cuff.

37. The catheter of claim 20, wherein the second section comprises a hub and a cuff, and the antimicrobial agent extends at least from the hub to the cuff.

38. The catheter of claim 20, wherein the second section comprises a cuff, and the inhibitory polymer comprises an antiproliferative and extends at least from the cuff to the venotomy.

39. The catheter of claim 20, wherein the first section terminates at a catheter tip, and the inhibitory polymer comprises an antithrombotic, a thrombolytics or fibrinolytic and is disposed at the catheter tip.

40. The catheter of claim 20, wherein the second section comprises a hub and a cuff, the antimicrobial agent extends at least from the hub to the cuff, and the inhibitory polymer comprises an antiproliferative that extends at least from the cuff to the venotomy and an antithrombotic, a thrombolytics or fibrinolytic that is disposed at the catheter tip.
